(19) United States
(12) Patent Application Publication
(10) Pub. No.: US 2004/0171624 A1
(43) Pub. Date: Sep. 2, 2004

(54) Pharmaceutical composition for treating mood disorders

(76) Inventors: Makoto Ozeki, Yokkaichi-shi (JP); Tsutomu Okubo, Yokkaichi-shi (JP); Lekh Raj Juneja, Yokkaichi-shi (JP)

Correspondence Address: BIRCH STEWART KOLASCH & BIRCH, PO BOX 747, FALLS CHURCH, VA, 22040-0747, US

(21) Appl. No.: 10/790,730

(22) Filed: Mar. 3, 2004

(30) Foreign Application Priority Data
Aug. 24, 2001 (JP) 2001-253740

(51) Int. Cl. A61K 31/52

(52) U.S. Cl. 514/263.4

(57) ABSTRACT

A pharmaceutical composition for treating or preventing mood disorders, comprising theanine. A food or beverage for ameliorating or preventing mood disorders, comprising theanine. The pharmaceutical composition and the food and beverage are safe and have a significant suppressive effect on the depression in a mood disorder.

### BACKGROUND OF THE INVENTION

1. Field of the Invention

The present invention relates to a pharmaceutical composition for treating or preventing mood disorders, comprising theanine. Also, the present invention relates to a food or beverage for ameliorating or preventing mood disorders, comprising theanine.

2. Discussion of the Related Art

Conventionally, mental disorders called “manic-depressive disorders” have been classified into disorders showing both a manic state and a depressed state (dipolar disorders) and disorders showing only a depressed state (monopolar disorders). However, manic-depressive disorders are now called “mood disorders” as a generic name referring to these two kinds of disorders. According to the results of epidemiological questionnaires conducted recently in Western advanced countries, the proportion of the number of individuals suffering from mood disorders especially suffering from depression or showing a depressed state to the total population (morbidity of individuals suffering from depression or showing a depressed state) has been certainly increasing. Especially with the coming of aging society, it is said that the morbidity exceeds 10%. Although the causations for mood disorders have not yet been clarified, it might be due to “the deficiency of function of a monoamine in the brain.” At present, based on this presumption, a medicament acting on the function of the monoamine in the brain is used for the treatment of depression in mood disorders.

The medicament exhibiting an antidepressive action includes an MAO inhibitor. MAO is an abbreviation standing for a monoamine oxidase, which is an enzyme acting to oxidize a chemical transmitter such as a monoamine including norepinephrine, serotonin or dopamine, thereby inactivating the chemical transmitter. The MAO inhibitor includes iproniazid, phenyprazine, phenelzine, nialamide, isocarboxyazid, safrazine and the like. However, there are many limitations upon its use because of problems of severe side effects such as hepatic disorders, the risk of the combined use of the inhibitor with a tricyclic antidepressant, limitations in the intake of high tyramine-content food, or the like. Therefore, all MAO inhibitors are discontinued to be sold at present.

The tricyclic antidepressant is often used as an antidepressant. Representative examples include imipramine hydrochloride (trade name: Tofranil), clomipramine hydrochloride (trade name: Anafranil), aminotriptin hydrochloride (trade name: Tryptanol), desipramine hydrochloride (trade name: Pertofrane), amoxapine (trade name: Amoxan), lofepramine hydrochloride (trade name: Amplit) and the like.

The action mechanism of the tricyclic antidepressant is an action of suppressing reuptake of a monoamine released from a nerve ending. The medicament having a strong suppressive effect on reuptake of a monoamine norepinephrine includes desipramine hydrochloride and amoxapine, and the medicament having a strong suppressive effect on reuptake of serotonin includes clomiprane hydrochloride. It is understood that aminotriptin hydrochloride and imipramine hydrochloride act moderately for suppressing reuptake of norepinephrine and serotonin.

A tetracyclic antidepressant has been developed as a medicament having smaller side effects than those of the tricyclic antidepressant. Representative examples include maprotiline hydrochloride (trade name: Ludiomil), mianserin hydrochloride (trade name: Tetramide) and setiptiline maleate (trade name: Tecipul). The action of the tetracyclic antidepressant is mainly an effect of accelerating release of a monoamine from a nerve ending. It is understood that the tetracyclic antidepressant has an effect of blocking α-2 receptors, thereby increasing the amount of a monoamine released.

The tricyclic and tetracyclic antidepressants have been known to have side effects. One of the side effects is an anti-choline symptom. It has been known that the tricyclic and tetracyclic therapeutic agents antagonize to an action of acetylcholine as a chemical transmitter in the parasympathetic nervous system. Concrete symptoms of the side effects include an increase in ocular tension by means of light scattering or the like, dry mouth, suppression of digestive tracts such as constipation, difficulty in urination, and the like. Besides the anti-choline symptom, drowsiness is induced by their suppressive action on the central nervous system, and further orhostatic hypotension has been also known as their side effect on the circulatory system. The side effect of the tetracyclic antidepressant is smaller than that of the tricyclic antidepressant, but its antidepressive effect is said to be smaller.

There are therapeutic agents called SSRIs (selective serotonin reuptake inhibitors) which are antidepressants having smaller side effects than those of the tricyclic and tetracyclic antidepressants. SSRIs have the feature of high selectivity for reuptake of serotonin among monoamines released from a nerve ending. A representative medicament is fluvoxamine maleate (trade names: Depromel and Luvox). Its side effects include digestive diseases such as nausea and emesis at an initial stage of administration, with the side effects being smaller than those of the tricyclic and tetracyclic antidepressants. In addition, its combined use with the MAO inhibitor, an anti-allergic agent such as terfenadine (trade name: Triludan) or astemizole (trade name: Hismanal), or a digestive movement improver such as cisapride (trade names: Acenalin and Risamol) has been known to be contraindicative.

In addition, a common feature of the antidepressants includes a delayed exhibition of their effects. It has been known that the antidepressants are highly fat-soluble and have a high binding ratio to plasma proteins, so that medical effectiveness is appeared slowly. Therefore, it is deduced that the effect is exhibited by some influences caused by continuous stimulation. Also, it has been reported that the number of receptors is decreased by continuous stimulation with monoamines released.

### SUMMARY OF THE INVENTION

An object of the present invention is to provide a composition for treating or preventing mood disorders. More specifically, an object of the present invention is to provide a composition for treating, ameliorating or preventing mood disorders, wherein the composition is capable of exhibiting its effect at an early stage without any side effects.

These and other objects of the present invention will be apparent from the following description.

As a result of intensive studies on substances effective in the treatment of mood disorders, the present inventors have found that theanine, an amino acid abundantly contained in green tea, is effective for solving the problems incurring in the conventional methods. The present invention has been accomplished thereby. The present inventors have found for the first time that theanine gives the above effect.

The present invention relates to:

(1) a pharmaceutical composition for treating or preventing mood disorders, comprising theanine;

(2) a food or beverage for ameliorating or preventing mood disorders, comprising theanine; and

(3) use of theanine for manufacture of the pharmaceutical composition of the above (1), or manufacture of the food or beverage of the above (2).

According to the present invention, the mood disorders of which symptom is mainly depression or a depressed state can be treated, ameliorated or prevented safely and effectively without worrying about side effects.

### BRIEF DESCRIPTION OF THE DRAWINGS

FIG. 1 is a graph showing a progressive change in an average score of the Hamilton scale in each of a group of patients administered with the theanine formulated-tablet and a group of patients administered with the control tablet.

FIG. 2 is a graph showing a progressive change in an average score of the Hamilton scale in each of a group of patients administered with the theanine formulated-tablet and a group of patients administered with the aminotriptin hydrochloride formulated-tablet.

### DETAILED DESCRIPTION OF THE INVENTION

The term “mood disorders” as used herein refers to symptoms given in International Classification of Diseases, 10th edition, issued by World Health Organization. According to the classification., the mood disorders are described as symptoms showing changes in moods such as depression and elation, and the fundamental disorder in the mood disorders is defined as follows: “A change in moods or emotions, which is usually changed to depression or to elation. Generally, this change in moods is accompanied by a change in whole activity, and most of other symptoms occur secondarily from this change or can be easily understood from the relationship therewith.” The mood disorders can be classified into disorders showing both a manic state and a depressed state (dipolar disorders) and disorders showing only a depressed state (monopolar disorders). In the mood disorders in both cases, the symptoms of depression or a depressed state are mainly observed.

The symptoms observed in patients in a depressed state include depressed moods, loss of interest and pleasure, an easily increased fatigue and a decreased activity due to a loss in vitality, feel of severe fatigue after trying hard, a diminished concentration and attentiveness, lowered self-evaluation and self-confidence, feelings of guilt and sense of worthlessness, hopeless and pessimistic views for future, ideas and gestures of self-injury and suicide, sleep disorders, loss of appetite and the like. In order to establish the diagnosis according to the diagnostic guidelines ICD-10 (World Health Organization; The ICD-10 Classification of Mental and Behavioural Disorders, WHO, Geneva, pp. 181-191, 1992), at least two items selected from depressed moods, loss of interest and pleasure, and easily increased fatigue, and at least two items selected from diminished concentration and attentiveness, lowered self-evaluation and self-confidence, feelings of guilt and sense of worthlessness, hopeless and pessimistic view for future, ideas and gestures of self-injury or suicide, sleep disorder, and loss of appetite must persist at least 2 weeks.

In the assessment of the symptoms of depression as above, a questionnaire referred to the Hamilton scale is generally used. The Hamilton scale consists of 21 items, i.e., “1. depressed mood,” “2. feelings of guilt,” “3. suicide,” “4. insomnia early,” “5. insomnia middle,” “6. insomnia late,” “7. work and activities,” “8. retardation: psychomotor,” “9. agitation,” “10. anxiety (psychological),” “11. anxiety (somatic),” “12. somatic symptoms (gastrointestinal),” “13. somatic symptoms general,” “14. genital symptoms,” “15. hypochondiasis,” “16. diminished insight,” “17. loss of weight,” “18. diurnal variation, worse in the morning or the evening,” “19. depersonalization and derealization,” “20. paranoid symptoms” and “21. obsessional and compulsive symptoms.” Depending on the extent of the severity of each symptom, the depression is rated under 5 ranks of “absent,” “mild or low,” “low-moderate,” “high-moderate” and “severe,” or under 3 ranks “absent,” “slight or doubtful” and “evident” depending on the kind of symptoms.

As the concrete method of rating the symptoms of depression by the Hamilton scale, reference can be made to, for instance, Max Hamilton, A Rating Scale for Depression, J. Neurol. Neurosurg. Psychiat. 23, 56-62 (1960).

The present invention provides a pharmaceutical composition for treating or preventing mood disorders, and a food or beverage for ameliorating or preventing mood disorders, wherein each contains theanine. The therapeutic effect or the like of the pharmaceutical composition or the like on the mood disorders is attributable to the action of theanine as an active ingredient. The term “treating,” “ameliorating” or “preventing” of mood disorders as used herein refers to treating, ameliorating or preventing at least one symptom of the mood disorders. The therapeutic or ameliorative effect of the pharmaceutical composition, the food or beverage of the present invention (may be also collectively referred to herein as “the composition”), or theanine on mood disorders can be confirmed, for instance, by assessing the symptoms of patients suffering from depression or individuals in a depressed state according to the assessment method by the Hamilton scale, for a case where the patients or individuals are allowed to take the composition or theanine and a case where the patients or individuals are not allowed to take it, and comparing the results of both cases. The preventing effect can be confirmed, for instance, by assessing the symptoms of individuals after the intake of the composition or theanine according to the assessment method by the Hamilton scale to regularly check the absence of incidence of symptoms of depression or a depressed state.

For example, in Test Example 1 set forth below, the therapeutic effect of the pharmaceutical composition of the present invention on mood disorders was confirmed in the manner described above. According to the results in Test Example mentioned above, the composition of the present invention is considered to be significantly effective for treating or ameliorating the mood disorders of which symptom is mainly symptom of depression or a depressed state.

Specifically, it is found in Test Example mentioned above that the pharmaceutical composition exhibits a therapeutic effect especially on the following items in the Hamilton scale: “1. depressed mood,” “2. feelings of guilt,” “3. suicide,” “8. retardation: psychomotor,” and “16. diminished insight.” These symptoms are observed characteristically in patients suffering from depression or showing a depressed state, and the symptoms are different from those of women in a depressed state caused before and during menstruation.

Therefore, the composition of the present invention is effective especially when the symptoms of mood disorders are at least one symptom selected from the group consisting of depressed mood, feelings of guilt, suicide, retardation: psychomotor, and diminished insight.

Theanine usable in the present invention is a glutamic acid derivative contained in tea-leaves, which is the main component of tastiness (umami) of tea. It is used as a food additive for seasoning. Processes for preparing theanine used in the present invention include, but are not limited to, a process of extracting theanine from tea-leaves with any extracting solvent; a process for obtaining theanine by an organic synthesis reaction [Chem. Pharm. Bull., 19(7), 1301-1307 (1971)]; a process of treating a mixture of glutamine and ethylamine with glutaminase to give theanine (Japanese Examined Patent Publication No. Hei 7-55154); a process comprising culturing cultured cells of tea in a medium containing ethylamine, thereby achieving growth promotion of the cultured cells while increasing the cumulative amount of theanine in the cultured cells (Japanese Patent Laid-Open No. Hei 5-123166); modification processes in which ethylamine is substituted by an ethylamine derivative such as ethylamine hydrochloride in the processes using cultured cells disclosed in Japanese Examined Patent Publication No. Hei 7-55154 or Japanese Patent Laid-Open No. Hei 5-123166; and the like, and any of the processes may be used. The above-mentioned “tea-leaves” include green tea-leaves, oolong tea-leaves, black tea-leaves, and the like. Theanine can be used as any of L-theanine, D-theanine and DL-theanine. Among them, the L-form is preferred in the present invention, because the L-form is approved as a food additive, and is economically utilizable. Also, if there is a commercially available product of theanine, such a product may be used. Incidentally, theanine used in the present invention may be in any forms, such as purified products, crudely purified products and extracts.

The theanine used in the present invention has high safety. For instance, in an acute toxic test using a mouse, there are no cases of death with an oral administration at 5 g/kg weight, and there are found no abnormalities in the general states, weight and the like. Also, especially L-theanine is known as a main component of umami (tastiness) of the green tea, and is also used as a food additive giving umami, without the limitation of its added amount under the regulation for food hygiene. Moreover, contrary to the conventional drugs, since there is no side effect by theanine at all, the mood disorders can be safely and effectively treated, ameliorated or the like according to the composition of the present invention.

The composition of the present invention includes, for instance, a medicament in the forms of tablets, capsules, powders, granules, beverages, and the like; foods such as dry foods and supplements; and beverages such as refreshing beverages, mineral waters, luxury beverages and alcoholic beverages, wherein each composition contains theanine. Incidentally, the same effects can be obtained as those of the composition of the present invention by using the theanine per se.

The pharmaceutical composition of the present invention is obtained by forming a preparation by combining theanine with a known pharmaceutical carrier. For instance, a preparation which is suitable for oral administration can be obtained by combining theanine with a pharmacologically acceptable liquid or solid carrier suitable for oral administration. Also, a solvent, a dispersant, an emulsifier, a buffer, a stabilizer, an excipient, a binder, a disintegrant, a lubricant, or the like is optionally added thereto, so that a solid agent such as a tablet, a capsule, a powder, a fine powder, or a granule, or a liquid agent such as a healthcare drink, a suspension agent or an emulsion agent can be formed. Furthermore, there can be also made into a dry product which can be made liquid by adding an appropriate liquid carrier before use.

The pharmaceutical carrier can be selected depending upon the administration form and preparation form of the pharmaceutical composition. In the case of an orally administered preparation comprising a solid composition, there can be utilized as a carrier, for instance, starch, lactose, saccharose, mannitol, carboxymethyl cellulose, cornstarch, an inorganic salt or the like. In addition, during the preparation of the orally administered preparation, a binder, a disintegrant, a surfactant, a lubricant, a fluidity accelerator, a flavor, a colorant, a perfume, or the like can be further formulated. In the case of forming into a tablet, for instance, the tablet may be covered with a sugar-coating made of sucrose, gelatin or hydroxypropyl cellulose, or with a film made of a substance soluble in the stomach or intestine as desired. In the case of an orally administered preparation comprising a liquid composition, for instance, purified water, ethanol or the like is utilized as a carrier. Furthermore, an auxiliary agent such as a wetting agent or a suspending agent, a sweetener, a flavor, an antiseptic, or the like may be added as desired.

The food or beverage of the present invention is obtained by formulating theanine during the preparation of a known food or beverage. During the preparation, optional ingredients as those listed below may be added in proper amounts so long as the exhibition of the desired effects of the present invention would not be hindered. The food and beverage as used herein are not strictly distinguishable, and also referred to as “foodstuff” from the above viewpoint.

For instance, the beverage of the present invention includes, but is not particularly limited to, teas such as green tea, oolong tea, black tea and herb tea, fruit juice concentrates, reconstituted juice concentrates, fresh juices, mixed fruit juices, fruit grain-containing fruit juice, fruit juice-containing beverages, mixed fruit/vegetable juice, vegetable juice, carbonated beverages, soft drinks, milk beverage, Japanese sake, beer, wine, cocktails, shochu, whiskey, and the like, wherein the beverage contains theanine.

Also, in the foodstuff of the present invention, crude medicines, herbs, amino acids, vitamins, and other materials and raw materials which are acceptable in foods and beverages may also be used together.

The above crude medicine includes, but are not particularly limited to, Common valerian, Angelica acutiloba, Paeonia lactiflora, peony, Panax ginseng and the like, which are effective for maintaining hormonal balance in women.

The above herbs include, but is not particularly limited to, anise, carrot seed, clove, coriander, cypress, cinnamon, juniper, ginger, sweet orange, pine needle, basil, patchouli, bitter orange, fennel, black pepper, bay, peppermint, bergamot, mandarin, myrrh, lemongrass, rosemary, grapefruit, cedarwood, citronella, sage, thyme, tea tree, violet leaf, vanilla, hyssop, eucalyptus, lime, lemon, ylang-ylang, cardamon, clary sage, jasmine, geranium, chamomile, Bulgarian rose, rose, olibanum, lavender, chamomile, geranium, sandalwood neroli, verbena, petigrain, vetiver, majoram, lemon balm (Melissa officinalis), rosewood, Hypericum, St. John's wort, and kawakawa, with preference given to peppermint, bergamot, ylang-ylang, geranium, chamomile, lavender, St. John's wort, and kawakawa, which have sedative and relaxation effects. The forms of these herbs include, but are not particularly limited to, extract, essential oil, dry powder, and the like.

The above amino acid includes, but are also not particularly limited to, for example, glutamine, glutamic acid, inosinic acid, alanine, arginine, aspartic acid, threonine, serine, γ-aminobutyric acid, taurine, thiotaurine, hypotaurine and the like.

The above vitamin includes, but is not limited to, vitamin A, vitamin B₁, vitamin B₂, vitamin B₆, vitamin B₁₂, vitamin C, vitamin D, vitamin E, vitamin K, folic acid, nicotinic acid, lipoic acid, pantothenic acid, biotin, ubiquinone and prostaglandin, as well as derivatives of these vitamins.

The above mineral includes, but is not limited to, calcium, iron, magnesium, copper, zinc, selenium, potassium, and the like.

In addition, in the preparation of the foodstuff of the present invention, there can be used aloe, royal jelly, melatonin, placenta, propolis, isoflavone, soybean lecithin, egg yolk lecithin, egg yolk oil, chondroitin, cacao mass, collagen, vinegar, cholera, spirulina, ginkgo leaves, green tea, tochu tea (Eucommia ulmoides), Chinese wolfberry tea, oolong tea, mulberry leaf, Rubus suavissimus (tencha), banaba tea, unsaturated fatty acids, saccharides such as oligosaccharides, bacteria such as bifidobacteria and red koji, mushrooms such as agaricus (Agaricus blazei), Agaricus blazei Murrill, reisi (ganoderma) and Grifloa frondosa, fruits such as blueberry, prune, grape, olive, Japanese apricot and citruses, seeds such as peanut, almond, sesame and pepper, vegetables such as green pepper, chili, Welsh onion, pumpkin, gourd, carrot, burdock, jute leaf (Corchorus capsularis), garlic, perilla, Japanese horseradish (wasabi), tomato, (pickled) shallot, leaf vegetables, tubers and beans, seaweeds such as wakame, fishes and shells, meat, poultry and whale meat, cereals and the like. Further, extracts, dried products, crudely purified products, purified products, processed products, fermentation products and the like of the above-mentioned components can be also used.

The foodstuff of the present invention is not particularly limited so long as the foodstuff is in the form that is orally administrable such as solution, suspension, powder, or molded solid product. Specifically, pasty products, processed soybean products, seasonings, mousses, jelly, frozen confectionaries, candies, chocolates, chewing gums, crackers, cakes, breads, soups, coffees, cocoas, teas, green tea, juices, milk beverages, dairy products, liquors, and the like.

The process for preparing the composition of the present invention includes, but is not particularly limited to, general processes of producing foodstuffs and pharmaceuticals, such as a process of mixing theanine with other starting materials in a powdery form, a process of dissolving theanine and other starting materials in a solvent to give a mixed solution, a process of lyophilizing the mixed solution, and the like.

The composition of the present invention is completed by realizing an inventive idea of use of theanine in the treatment or the like of mood disorders on the basis of a newly found pharmacological effect of theanine. From this viewpoint, in one embodiment of the present invention, there can be also provided use of theanine for manufacture of the composition of the present invention.

The content of theanine in the composition of the present invention is not particularly limited as long as theanine can be administered preferably in the following range of dose in view of the administration form, use and the like. In consideration of the effective dose of theanine, the content of theanine is usually preferably 0.00025% by weight or more, more preferably from 0.0025 to 100% by weight. Additionally, in consideration of usual forms of the composition of the present invention (for example, the forms of the above solid foods, liquid foods and pharmaceuticals), the content is more preferably from 0.08 to 99% by weight.

The dose of the composition of the present invention, especially the pharmaceutical composition, can be varied and determined suitably depending upon the administration form, administration style, the purpose of use, age, weight and symptoms of an individual to be administered. Generally, the composition of the present invention may be administered such that the dose of theanine in the present invention is preferably from 0.2 to 200 mg/kg body weight, more preferably from 0.5 to 50 mg/kg body weight to human (for example, adults) per day. Since the effects can be sufficiently exhibited by administering theanine in this range, the above dose range of theanine can be said to be an effective dose range of theanine. However, since there are individual differences in the kind and extent of symptoms listed above, the dose of the composition of the present invention or theanine is not limited to the above range.

The pharmaceutical composition of the present invention can be administered in a single dose or in dose of several divided portions within one day in an amount in the desired dose range, and a dose period can be arbitrary.

### EXAMPLES

The present invention will be describe hereinbelow by means of the following Examples and Test Examples, without intending to limit the present invention thereto.

### Preparation Example 1

### Preparation of Theanine by Enzymatic Method

The amount 21.9 g of glutamine and 28.5 g of ethylamine hydrochloride were allowed to react at 30° C. for 22 hours in 0.5 L of 0.05 M borate buffer (pH 9.5) in the presence of 0.3 U glutaminase (manufactured by Amano Enzyme Inc.). Thereafter, the reaction mixture was subjected to column chromatography using Dowex 50×8 column and Dowex 1×2 column (both manufactured by Muromachi Kagaku Kogyo K.K.), and thereafter the resulting product was treated with ethanol, thereby isolating a desired product from the reaction mixture.

The identification of the obtained substance as L-theanine was carried out by subjecting the isolated substance to amino acid analyzer and paper chromatography, whereby confirming that the isolated substance exhibits the same behaviors as the standard substance. Moreover, when the isolated substance was subjected to hydrolysis treatment with hydrochloric acid or glutaminase, glutamic acid and ethylamine were generated at a molar ratio of 1:1. Since the isolated substance was hydrolyzed by glutaminase, it was shown that ethylamine was bonded at the y-position of glutamic acid. In addition, it was also confirmed by using the glutamic acid dehydrogenase that glutamic acid generated by hydrolysis had an L-form. From the above, the resulting isolated substance was finally confirmed to be L-theanine. By the above procedure, 8.5 g of L-theanine was obtained.

### Preparation Example 2

### Extraction of L-Theanine from Tea Leaves

Ten kilograms of tea leaves (Camellia sinensis L.) were subjected to extraction with boiling water. The resulting extract was then applied to a cationic exchange resin (“Dowex HCR W-2,” manufactured by Muromachi Kagaku Kogyo K.K.), and the component adsorbed to the resin was eluted with 1 N NaOH. The eluted fraction was applied to an activated carbon (“Taiko Kasseitan SG” manufactured by Futamura Kagaku Kogyo K.K.), and eluted with 15% ethanol. The resulting eluted fraction was concentrated with an RO membrane (“NTR 729 HF” manufactured by NITTO DENKO CORPORATION). Thereafter, the concentrate was purified by column chromatography in the same manner as in Preparation Example 1. Furthermore, the purified product was recrystallized, to give 24.8 g of L-theanine.

Here, in the preparation of each composition described hereinbelow, L-theanine [trade name: Suntheanine, manufactured by Taiyo Kagaku Co., Ltd.] was used.

### Example 1

### Preparation of Theanine-Formulated Tablet

As one example of the composition of the present invention, a theanine-formulated tablet was prepared by mixing the raw materials given below, and tabletting the resulting mixture.

| | | | |
|---|---|---|---|
| Frosted Sugar | 71.67% | by weight | (0.5375 g) |
| Trehalose | 10% | by weight | (0.075 g) |
| L-Theanine | 13.33% | by weight | (0.1 g) |
| Sucrose Fatty Acid Ester | 1% | by weight | (0.0075 g) |
| Flavor (Lemon Flavor) | 4% | by weight | (0.03 g) |
| Total | 100% | by weight | (0.75 g) |

Specifically, each of the raw materials was mixed in accordance with the above composition, and the mixture was granulated to give a tablet of 0.75 g.

### Comparative Example 1

### Preparation of Control Tablet

A control tablet was prepared by mixing the raw materials given below, and tabletting the resulting mixture.

| | | | |
|---|---|---|---|
| Frosted Sugar | 85% | by weight | (0.6375 g) |
| Trehalose | 10% | by weight | (0.075 g) |
| Sucrose Fatty Acid Ester | 1% | by weight | (0.0075 g) |
| Flavor (Lemon Flavor) | 4% | by weight | (0.03 g) |
| Total | 100% | by weight | (0.75 g) |

Specifically, each of the raw materials was mixed in accordance with the above composition, and the mixture was granulated to give a tablet of 0.75 g.

### Example 2

### Preparation of Theanine-Formulated Candy

As one example of the composition of the present invention, a theanine-formulated candy was prepared using the following raw materials.

| | | |
|---|---|---|
| Granulated Sugar | 64 | kg |
| Malt Syrup | 23 | kg |
| L-Theanine | 10 | kg |
| Flavor (Lemon Flavor) | 0.05 | kg |
| 50% Tartaric Acid | 1 | kg |
| Water | 30 | kg |

The granulated sugar was dissolved in 20 kg of water with heating to 110° C. Ten kilograms of the remaining water in which L-theanine was previously dissolved, and malt syrup were added thereto, and the temperature was raised to 145° C. After heating was stopped, the flavor and 50% tartaric acid were added thereto and mixed. The mixture was cooled to 75° to 80° C., and formed with a molding roller, to give a theanine-formulated candy (1.2 g per drop). The content of L-theanine in the candy was determined by HPLC. As a result, its content was 89.6 mg/g (7.47% by weight) per drop of 1.2 g.

### Example 3

### Preparation of Theanine-Formulated Blueberry Beverage

As one example of the composition of the present invention, a theanine-formulated blueberry beverage was prepared using the following raw materials.

| Fructose Sucrose Solution | 12 | kg |
|---|---|---|
| Blueberry Concentrate Juice | 1 | kg |
| 1/5 Transparent Lemon Juice | 0.4 | kg |
| Sodium Citrate | 0.05 | kg |
| 50% Sodium Citrate (Crystals) | for pH | |
| | adjustment | |
| L-Theanine | 0.1 | kg |
| Flavor (Blueberry Flavor) | 0.05 | kg |
| Water | Proper | |
| | amount | |
| Total | 100 | kg |

Fructose glucose solution, blueberry concentrate juice, ⅕ transparent lemon juice, sodium citrate and L-theanine were added to water to dissolve the components with stirring. The solution was adjusted to pH 3.1 with 50% sodium citrate (crystals) and heated to 95° C., and the flavor was added thereto, and the mixture was filled into a 100-ml can and then cooled to produce a theanine-formulated blueberry beverage. The L-theanine in the blueberry beverage was quantified. As a result, the content was 98.3 mg/100 ml (0.098% by weight).

### Example 4

### Preparation of Theanine-Formulated Grapefruit Beverage

As one example of the composition of the present invention, a theanine-formulated grapefruit beverage was prepared using the following raw materials.

| Fructose Sucrose Solution | 6 | kg |
|---|---|---|
| L-Theanine | 0.1 | kg |
| Ferric Pyrophosphate | 0.06 | kg |
| Placenta Extract | 0.01 | kg |
| 100% Grapefruit Juice | 30 | kg |
| Sodium Citrate | for pH | |
| | adjustment | |
| Flavor (Grapefruit Flavor) | 0.05 | kg |
| Water | Proper | |
| | amount | |
| Total | 100 | kg |

Fructose sucrose solution, L-theanine, ferric pyrophosphate, placenta extract and 100% grapefruit juice were added to water with stirring to dissolve the components. The pH of the resulting solution was adjusted to 3.1 by using sodium citrate. After the temperature was raised to 95° C., the flavor was added thereto. The resulting solution was filled and then cooled to produce a theanine-formulated grapefruit beverage. The L-theanine in the grapefruit beverage was quantified. As a result, its content was 96.4 mg/100 ml (0.096% by weight).

### Test Example 1

### Evaluation Test for Therapeutic Effects on Mood Disorders

The therapeutic effects of the pharmaceutical composition of the present invention on mood disorders were examined for a group of 24 normofolatemic patients (average age: 42-year old). The normofolatemic patients refer to those individuals showing a normal folate plasma level (3 to 17 ng/ml), who are not patients with neural tube defect caused by lack of folate.

In diagnosis, the patients were assessed to be mild to severe according to the diagnostic criteria of DMS III R (Diagnostic and Statistical Manual of Mental Disorders, 3rd Ed. Rev American Psychiatric Association, Washington D.C., pp. 235-253, 1987). The test was conducted in double blind, and the test period was 3 weeks. The 24 patients (average body weight: 61 kg) were divided into two even groups according to the above diagnostic results. One group was administered with the theanine-formulated tablet prepared in Example 1 (group administered with the theanine-formulated tablet), and the other group was administered with the control tablet prepared in Comparative Example 1 (group administered with the control tablet). Each patient was administered with one tablet twice a day, at 10 am and 4 pm (amount of theanine intake: 200 mg). The therapeutic effects were assessed according to the Hamilton scale consisting of 21 items regarding the assessment for depression. The assessment was made before the intake of each tablet (i.e. before the beginning of the test) and on Day 7, Day 14 and Day 21 from the intake, during each of the days, respectively.

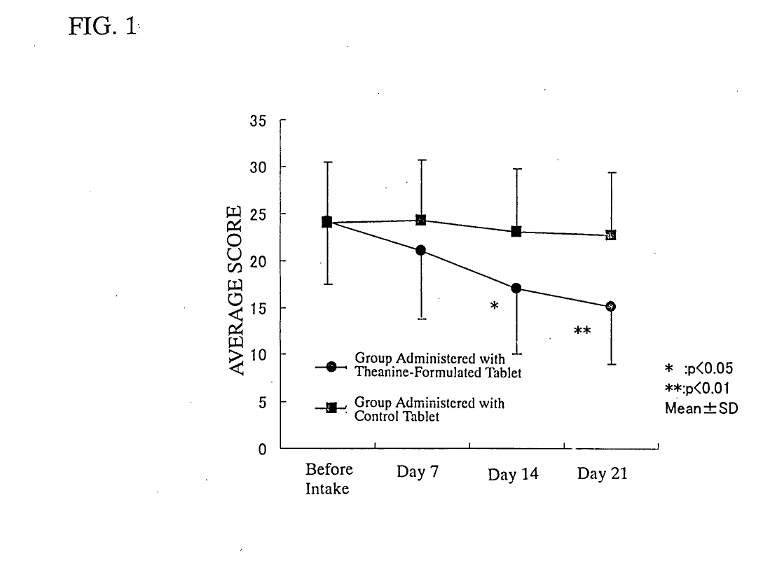

A progressive change in the average score of the Hamilton scale for each of the group of the patients administered with the theanine-formulated tablet of Example 1 and the group administered with the control tablet of Comparative Example 1 is shown in FIG. 1. The average score of the Hamilton scale was significantly decreased from “24” before the intake to “15” after 3 weeks of the intake in the group administered with the theanine-formulated tablet of Example 1 (p<0.01), while a reduction in the score in the group administered with the control tablet of Comparative Example 1 was hardly recognized. By the intake of the theanine-formulated tablet of Example 1, the therapeutic effects were observed as early as after 1 week from the intake.

The average score for each symptom item of the Hamilton scale in each group of patients before the intake and on Day 7, Day 14 and Day 21 from the intake is shown in Table 1. The average score was subjected to Student paired t-test against the score before the intake.

TABLE 1

| | Group Administered with Theanine-Formulated Tablet | | | |
|---|---|---|---|---|
| Items | Before Intake | Day 7 | Day 14 | Day 21 |
| 1. Depressed Mood | 2.833 ± 0.322 | 2.000 ± 0.389** | 1.083 ± 0.336** | 0.583 ± 0.288** |
| 2. Feelings of Guilt | 2.667 ± 0.333 | 2.000 ± 0.369* | 1.250 ± 0.351** | 0.833 ± 0.322** |
| 3. Suicide | 2.750 ± 0.372 | 2.000 ± 0.369* | 1.167 ± 0.322** | 0.667 ± 0.333** |
| 4. Insomnia Early | 0.333 ± 0.142 | 0.583 ± 0.149 | 0.583 ± 0.163 | 0.583 ± 0.193 |
| 5. Insomnia Middle | 0.250 ± 0.131 | 0.333 ± 0.142 | 0.500 ± 0.195 | 0.583 ± 0.193 |
| 6. Insomnia Late | 0.250 ± 0.131 | 0.250 ± 0.131 | 0.667 ± 0.188 | 0.583 ± 0.149* |
| 7. Work and Activities | 0.917 ± 0.260 | 1.167 ± 0.241 | 0.917 ± 0.229 | 1.000 ± 0.213 |
| 8. Retardation: Psychomotor | 2.833 ± 0.207 | 1.917 ± 0.288** | 1.250 ± 0.351** | 0.833 ± 0.345** |
| 9. Agitation | 0.917 ± 0.193 | 1.000 ± 0.174 | 1.000 ± 0.174 | 0.917 ± 0.149 |
| 10. Anxiety(Psychological) | 0.833 ± 0.241 | 0.833 ± 0.207 | 1.000 ± 0.213 | 1.000 ± 0.213 |
| 11. Anxiety Somatic | 0.833 ± 0.297 | 0.750 ± 0.279 | 0.833 ± 0.271 | 0.750 ± 0.218 |
| 12. Somatic Symptoms | 0.417 ± 0.193 | 0.500 ± 0.195 | 0.500 ± 0.151 | 0.667 ± 0.142 |
| (Gastrointestinal) | | | | |
| 13. Somatic Symptoms | 0.917 ± 0.260 | 0.833 ± 0.241 | 0.583 ± 0.193 | 0.583 ± 0.193 |
| General | | | | |
| 14. Genital Symptoms | 0.417 ± 0.229 | 0.500 ± 0.230 | 0.417 ± 0.229 | 0.500 ± 0.230 |
| 15. Hypochondriasis | 1.333 ± 0.256 | 1.250 ± 0.279 | 1.250 ± 0.279 | 1.167 ± 0.241 |
| 16. Diminished Insight | 1.667 ± 0.188 | 1.083 ± 0.260* | 0.417 ± 0.229** | 0.333 ± 0.225** |
| 17. Loss of Weight | 1.083 ± 0.193 | 1.000 ± 0.246 | 1.000 ± 0.213 | 0.917 ± 0.193 |
| 18. Diurnal Variation - | 0.500 ± 0.195 | 0.583 ± 0.193 | 0.417 ± 0.149 | 0.500 ± 0.151 |
| Morning or Evening | | | | |
| 19. Depersonalization and | 0.833 ± 0.207 | 0.750 ± 0.218 | 0.750 ± 0.218 | 0.750 ± 0.218 |
| Derealization | | | | |
| 20. Paranoid Symptoms | 1.083 ± 0.260 | 1.083 ± 0.229 | 0.833 ± 0.241 | 0.750 ± 0.218 |
| 21. Obsessional and | 0.500 ± 0.195 | 0.583 ± 0.193 | 0.583 ± 0.193 | 0.583 ± 0.149 |
| Compulsive Symptoms | | | | |
| | Group Administered with Control Tablet | | | |
| Items | Before Intake | Day 7 | Day 14 | Day 21 |
| 1. Depressed Mood | 2.833 ± 0.241 | 2.333 ± 0.188 | 2.000 ± 0.302 | 2.417 ± 0.193 |
| 2. Feelings of Guilt | 2.167 ± 0241 | 2.250 ± 0.250 | 2.417 ± 0.229 | 2.083 ± 0260 |
| 3. Suicide | 1.917 ± 0.358 | 1.833 ± 0.366 | 2.083 ± 0.260 | 2.083 ± 0.229 |
| 4. Insomnia Early | 0.250 ± 0.179 | 0.333 ± 0.188 | 0.250 ± 0.179 | 0.250 ± 0.179 |
| 5. Insomnia Middle | 0.167 ± 0.167 | 0.083 ± 0.083 | 0.167 ± 0.112 | 0.083 ± 0.083 |
| 6. Insomnia Late | 0.083 ± 0.083 | 0.167 ± 0.112 | 0.250 ± 0.131 | 0.167 ± 0.112 |
| 7. Work and Activities | 1.917 ± 0.149 | 1.917 ± 0.193 | 1.583 ± 0.149 | 1.750 ± 0.131 |
| 8. Retardation: Psychomotor | 2.250 ± 0.218 | 2.083 ± 0.260 | 1.917 ± 0.313 | 2.083 ± 0.336 |
| 9. Agitation | 0.500 ± 0.195 | 0.667 ± 0.225 | 0.583 ± 0.229 | 0.583 ± 0.260 |
| 10. Anxiety(Psychological) | 1.083 ± 0.260 | 1.083 ± 0.260 | 0.833 ± 0.241 | 1.000 ± 0.246 |
| 11. Anxiety Somatic | 1.167 ± 0.241 | 1.000 ± 0.246 | 0.833 ± 0.241 | 0.750 ± 0.250 |
| 12. Somatic Symptoms | 0.417 ± 0.193 | 0.583 ± 0.229 | 0.583 ± 0.229 | 0.333 ± 0.188 |
| (Gastrointestinal) | | | | |
| 13. Somatic Symptoms | 0.417 ± 0.193 | 0.333 ± 0.188 | 0.417 ± 0.193 | 0.333 ± 0.188 |
| General | | | | |
| 14. Genital Symptoms | 0.500 ± 0.195 | 0.333 ± 0.188 | 0.333 ± 0.188 | 0.417 ± 0.193 |
| 15. Hypochondriasis | 1.750 ± 0.218 | 1.500 ± 0.230 | 1.500 ± 0.195 | 1.417 ± 0.229 |
| 16. Diminished Insight | 1.500 ± 0.151 | 1.197 ± 0.193 | 1.417 ± 0.193 | 1.417 ± 0.193 |
| 17. Loss of Weight | 1.083 ± 0.229 | 1.000 ± 0.246 | 1.000 ± 0.246 | 1.083 ± 0.229 |
| 18. Diurnal Variation - | 0.833 ± 0.241 | 1.000 ± 0.246 | 0.917 ± 0.229 | 0.917 ± 0.229 |
| Morning or Evening | | | | |
| 19. Depersonalization and | 1.250 ± 0.279 | 1.250 ± 0.218 | 1.417 ± 0.229 | 1.333 ± 0.188 |
| Derealization | | | | |
| 20. Paranoid Symptoms | 1.833 ± 0.297 | 1.750 ± 0.250 | 1.500 ± 0.195 | 1.167 ± 0.207 |
| 21. Obsessional and | 0.750 ± 0.218 | 0.833 ± 0.271 | 1.000 ± 0.213 | 1.000 ± 0.275 |
| Compulsive Symptoms | | | | |

Student paired t-test: vs. before intake
*p < 0.05
**p < 0.01

As shown in Table 1, significant amelioration in the symptoms “1. depressed mood,” “2. feelings of guilt,” “3. suicide,” “8. retardation: psychomotor,” and “16. diminished insight” were recognized on Day 7, Day 14 and Day 21 in the group administered with the theanine-formulated tablet. These symptoms are characteristics of the patients with depression or in a depressed mood. On the other hand, no amelioration in every symptom was observed in the group administered with the control tablet.

According to the above test results, the therapeutic effects of the pharmaceutical composition of the present invention on mood disorders were recognized. During the period of intake, records were also taken for occurrence of any side effects. However, no side effects were observed.

### Test Example 2

### Comparison to Tricyclic Antidepressant

Comparison of the therapeutic effects on mood disorders between a tricyclic antidepressant and the pharmaceutical composition of the present invention was made in a group of 10 normofolatemic patients (average age: 48-year old). In diagnosis, the patients were judged to be mild to severe according to the diagnostic criteria of DMS III R. The test was conducted in double blind, and the test period was 4 weeks. The 10 patients (average body weight: 55 kg) were divided into two even groups. One group was administered with the theanine-formulated tablet prepared in Example 1 (group administered with the theanine-formulated tablet), and the other group was administered with a tricyclic antidepressant aminotriptin hydrochloride-formulated tablet (group administered with the aminotriptin hydrochloride-formulated tablet). Each patient was administered with one tablet twice a day, at 10 am and 4 pm (amount of theanine intake: 200 mg; amount of aminotriptin hydrochloride intake: 50 mg). The therapeutic effects were evaluated according to the Hamilton scale consisting of 21 items regarding assessment for depression. The assessment was made before the intake of each tablet and on Day 14 and Day 28 after the intake, during each of the days. In addition to the Hamilton scale, side effects were recorded in a questionnaire.

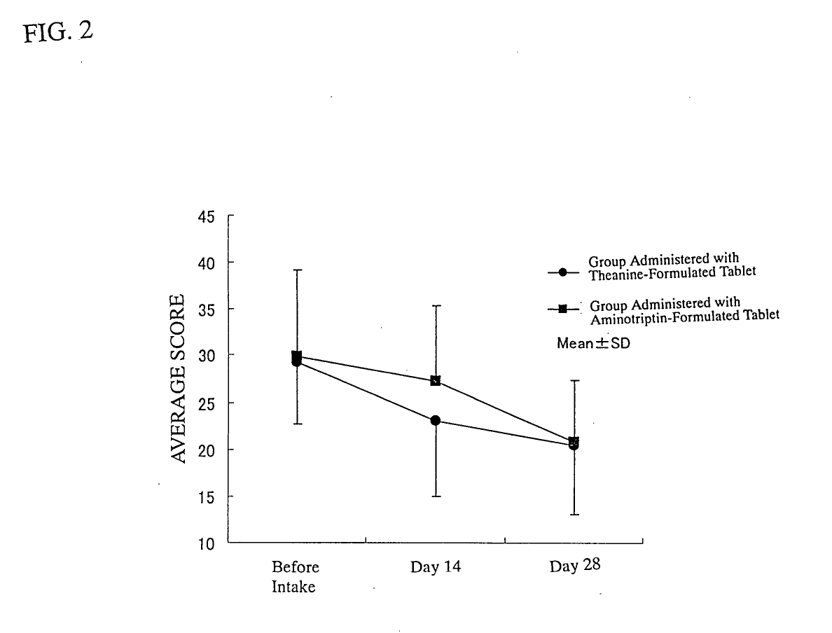

A progressive change in the average score of the Hamilton scale for each of the group of patients administered with the theanine-formulated tablet of Example 1 and the group administered with the aminotriptin hydrochloride-formulated tablet is shown in FIG. 2. The average score in the Hamilton scale was significantly decreased from “29” before the intake to “23” on Day 14 or to “20” on Day 27 in the group administered with the theanine-formulated tablet in Example 1. In the group administered with the aminotriptin hydrochloride-formulated tablet, on the other hand, the score was decreased to “27” on Day 14 so that the effects were hardly recognized, but the score was then decreased to “20” on Day 27. As described above, it was confirmed that the therapeutic effects of the pharmaceutical composition of the present invention were exhibited more rapidly.

The records of side effects in each tested individual during the test period are as follows.

Theanine-Formulated Tablet of Example 1

| Patient No. | Side Effects (Day 14) | Side Effects (Day 28) |
|---|---|---|
| A | None | None |
| B | None | None |
| C | None | None |
| D | None | None |
| E | None | None |

Aminotriptin Hydrochloride-Formulated Tablet

| Patient No. | Side Effects (Day 14) | Side Effects (Day 28) |
|---|---|---|
| F | Drowsiness | Drowsiness, Constipation |
| G | Dry Mouth | Dry Mouth |
| H | None | None |
| I | Constipation | Constipation |
| J | Palpitations, Constipation | Palpitations, Constipation |

During the test period, no side effects were recognized in all the individuals administered with the theanine-formulated tablet of Example 1, but side effects were recognized in 4 out of 5 individuals administered with the aminotriptin hydrochloride-formulated tablet.

### Test Example 3

### Evaluation Test for Ameliorative Effects on Mood Disorders

The evaluation test for ameliorative effects on mood disorders was conducted with the theanine-formulated candy of Example 2, the theanine-formulated blueberry beverage of Example 3 and the theanine-formulated grapefruit beverage of Example 4.

The test was conducted for a group of 9 normofolatemic patients (average age: 51-year old). In the diagnosis, the patients were judged to be mild to severe according to the diagnostic criteria of DMS III R. The test period was 4 weeks. The 9 patients (average body weight: 63 kg) were divided into groups, each consisting of 3 patients. Each patient was administered with one theanine-formulated candy of Example 2, one can of the theanine-formulated blueberry beverage of Example 3, or one can of the theanine-formulated grapefruit beverage of Example 4, twice a day, at 10 am and 4 pm (amount of theanine intake: about 200 mg). The therapeutic effects were evaluated according to the Hamilton depression scale consisting of 21 items regarding assessment for depression. The assessment was made before the intake of each composition and on Day 28 after the intake, during each of the days.

A progressive change of the score of the Hamilton scale for each patient during the test period is as follows:

Theanine-Formulated Candy of Example 2

| Patient No. | Before Intake | Day 28 |
|---|---|---|
| A | 30 | 21 |
| B | 24 | 19 |
| C | 26 | 24 |

Theanine-Formulated Blueberry Beverage of Example 3

| Patient No. | Before Intake | Day 28 |
|---|---|---|
| D | 38 | 30 |
| E | 22 | 20 |
| F | 35 | 33 |

Theanine-Formulated Grapefruit Beverage of Example 4

| Patient No. | Before Intake | Day 28 |
|---|---|---|
| D | 38 | 30 |
| E | 22 | 20 |
| F | 35 | 33 |

It can be seen that the score in each patient is decreased by all of the food and beverages in Examples 2 to 4, thereby revealing that mood disorders are ameliorated. Accordingly, the ameliorative effects of the food and beverage of the present invention on mood disorders were confirmed by this test.

According to the present invention, there are provided a safe pharmaceutical composition, and food and beverage, each having a significant suppressive effect on the depression in a mood disorder.

The present invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

What is claimed is:

1. A pharmaceutical composition for treating or preventing mood disorders, comprising theanine.

2. The pharmaceutical composition according to claim 1, wherein a symptom of mood disorders is at least one symptom selected from the group consisting of depressed mood, feelings of guilt, suicide, retardation: psychomotor, and diminished insight according to the assessment method by the Hamilton scale.

3. A food or beverage for ameliorating or preventing mood disorders, comprising theanine.

4. Use of theanine for manufacture of the pharmaceutical composition of claim 1 or 2, or manufacture of the food or beverage of claim 3.
